(19) United States
(12) Patent Application Publication
(10) Pub. No.: US 2008/0176936 A1
(43) Pub. Date: Jul. 24, 2008

(54) Classes of Compounds that Interact with Integrins

(76) Inventors: Wim Meutermans, Queensland (AU); Michael L. West, Queensland (AU); Giang Thanh Le, Queensland (AU); Judy Halliday, Queensland (AU); Christopher Clark, Queensland (AU)

Correspondence Address: MORRISON & FOERSTER LLP, 755 PAGE MILL RD, PALO ALTO, CA, 94304-1018, US

(21) Appl. No.: 11/813,737

(22) PCT Filed: Feb. 2, 2006
(86) PCT No.: PCT/AU2006/000129

§ 371 Date: Jul. 11, 2007

(30) Foreign Application Priority Data
Feb. 4, 2005 (AU) 2005900499

(51) Int. Cl. A61K 31/351; C07D 315/00; A61P 3/10

(52) U.S. Cl. 514/460; 549/418

(57) ABSTRACT

A method of inhibiting or effecting the activity of an integral receptor which comprises contacting an integrin with a compound of formula I, or a pharmaceutically acceptable salt thereof; General Formula I Wherein the ring may be of any configuration; Z is sulphur, oxygen, CH₂, NH, NR^{A} or hydrogen, in the case where Z is hydrogen then R¹ is not present, R^{A} is selected from the set defined for R₁ to R₅, X is oxygen or NR^{A} providing that at least one X of General Formula I is NR^{A}, X may also combine independently with one of R₁ to R₅ to form an azide, R1 to R5 are independently selected from the group comprising H, —(CO)R₆ or an alkyl, acyl, alkenyl, alkynyl, heteroalkyl, aryl, heteroaryl, arylalkyl or heteroarylalkyl substituent of 1 to 20 atoms, which is optionally substituted, and can be branched or linear wherein substituents may optionally be further substituted, wherein R₆ is selected from the group comprising an alkyl, acyl, alkenyl, alkynyl, heteroalkyl, aryl, heteroaryl, arylalkyl or heteroarylalkyl substituent of 1 to 20 atoms, which is optionally substituted, and can be branched or linear wherein substituents may optionally be further substituted, with the proviso that XR₂ or XR₃ or XR₄ or XR₅ is not NH₂, with the further proviso that not more than one of R2 to R5 is hydrogen, where the group X is NR^{A} and R^{A} is not hydrogen, the groups R^{A} and the corresponding group R₂ to R₅ may combine to form a cycle.

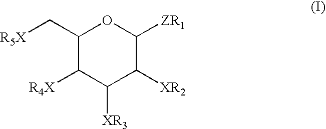

### FIELD OF THE INVENTION

The invention provides classes of biologically active compounds that interact in a pharmaceutically significant manner with integrin receptors.

### BACKGROUND OF THE INVENTION

The drug discovery landscape has been transformed by the genomics revolution. Advances in the understanding of biomolecular pathways and the roles they play in disease will lead to vast numbers of targets for therapeutic intervention. Integrins are a family of cell surface receptors that mediate cellular interactions with the extracellular matrix, with some integrins also involved in critical cell-cell adhesions. Integrins are composed of α and β transmembrane subunits selected from among 18α and 8β subunits. These subunits heterodimerize to produce at least 24 different receptors. The α and β subunits are also subject to alternate splicing and post-translational modifications, providing further structural diversity¹. Integrin mediated adhesive interactions are intimately involved in the regulation of many cellular functions including, embryonic development, tumour cell growth and metastasis, angiogenesis, programmed cell death, haemostasis, leukocyte homing and activation, bone resorption, clot retraction, and the response of cells to mechanical stress².

Considering the rate of generation and nature of the targets currently being deconvoluted by biologists, there is a need for the development of drug candidates, designed in a rational manner to purposely interact with selected targets, such as the integrins.

From a drug discovery perspective, carbohydrate pyranose and furanose rings and their derivatives are well suited as templates. Each sugar represents a three-dimensional scaffold to which a variety of substituents can be attached, usually via a scaffold hydroxyl group, although occasionally a scaffold carboxyl or amino group may be present for substitution. By varying the substituents, their relative position on the sugar scaffold, and the type of sugar to which the substituents are coupled, numerous highly diverse structures are obtainable.

An important feature to note with carbohydrates, is that molecular diversity is achieved not only in the type of substituents, but also in the three dimensional presentation. The different stereoisomers of carbohydrates that occur naturally, offer the inherent structural advantage of providing alternative presentation of substituents.

Nicolaou et. al. (Tetrahedron, 1997, 53, 8751-8778) have reported the synthesis and biological evaluation of a series of compounds which are purported to bind integrin receptors. The compounds of the current invention differ in two significant ways from those reported in the Nicolaou publication. In the first instance, the compounds of the current invention contain a nitrogen directly attached to the carbohydrate scaffold ring, whereas the Nicolaou compounds contain only oxygen. Additionally, the Nicolaou publication states on page 8760 that the compounds in this publication do not bind to the αᵥβ₃ or α_{llb}β₃ integrin receptors, in stark contrast to the affinity and selectivity demonstrated in the compounds of the current invention.

More recently, Kessler et. al., (Angew. Chemie., Int. Ed. Engl., 2000, 39 pp 2761-2764) have used carbohydrates, specifically glucuronic acids as amino acid surrogates in the synthesis of cyclic peptidomimetics to inhibit Integrins. This work takes quite a different approach to the compounds of the current invention in that the sugars are incorporated into a peptidic chain. Kessler et. al., (Angew. Chem., 2001, 113, pp. 3988-3991), have also reported the use of mannose as a scaffold for the preparation of integrin inhibitors. This work is similar to that of Nicolaou et. al., vide supra, and differs from the current invention in that there are no nitrogen atoms attached to the carbohydrate ring and the activity of the compounds is extremely low, being tested at 5 millimolar concentration (page 3991 table 1) as compared to the compounds of the current invention which were tested at 250 micromolar concentration.

Moitessier et. al., (Bioorg. Med. Chem., 2001, 9, pp 511-523) have reported a similar approach to that of Nicolaou and Kessler, this time using Xylose as the scaffold for compound preparation. Again, the compounds do not contain a nitrogen directly attached to the carbohydrate ring and exhibit only modest activity at 4 millimolar concentrations (page 515).

In a patent application by Kunz et. al, (WO99/07718), there is some overlap with compounds of the current invention, specifically when the 2 position of the sugar scaffold is substituted with a nitrogen. There is however, no specific or general exemplification of any compound with a nitrogen directly substituted to the carbohydrate ring, even in the 2 position. The methods proposed in the examples are further, not applicable to the case where the 2 position or any other position is an amino group. Further there is no evidence of biological affinity to integrins or indeed to any other biological receptor.

Employing a related methodology, Hirschmann et al (Hirschmann, J. Am. Chem. Soc., 1992, 114, 9217-9218; J. Am. Chem. Soc., 1993, 115, 12550-12568; J. Med. Chem., 1997, 41, 1382-1391) have designed and prepared carbohydrate based compounds against somatostatin receptors. These compounds show respectable activity in biological assays. The compounds disclosed do not however, contain an amino function directly attached to the carbohydrate ring and were not designed or tested to inhibit the integrin receptors. Hirschmann et al have sought patent protection (U.S. Pat. No. 5,552,534, U.S. Pat. No. 5,811,512; U.S. Pat. No. 6,030,942; WO 97/28172; WO 95/11686; WO 93/17032) in each of the cited patents or patent applications, the compounds do not disclose, exemplify or contemplate amino-substituted carbohydrates. Further the compounds disclosed are targeted to G-protein coupled receptors and integrins are not contemplated or exemplified. The compounds and methods disclosed are manifestly distinct from this present invention.

Thus there is a need for compounds which effectively bind or interact with integrin receptors. The present invention overcomes or at least partially overcomes the deficiencies in the prior art and provides compounds which effectively bind or interact with integrin receptors.

Using the axioms of this drug discovery methodology, we synthesised several novel classes of chemotypes in an effort to develop drug candidates against integrin targets. In each case the compounds are derivatives of amino-substituted carbohydrate rings. It is believed that the presence of at least one nitrogen at an X position on the scaffold increases the restriction of the rotation of the appended group, thereby providing enhanced bioactivity of the compound.

It will be clearly understood that, if a prior art publication is referred to herein, this reference does not constitute an admission that the publication forms part of the common general knowledge in the art in Australia or in any other country.

### SUMMARY OF THE INVENTION

In one aspect the invention provides a method of inhibiting or effecting the activity of an integrin receptor which comprises contacting an integrin with a compound of formula I, or a pharmaceutically acceptable salt thereof;

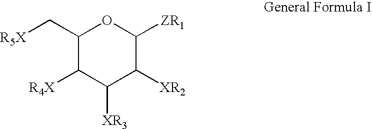

Wherein the ring may be of any configuration;

Z is sulphur, oxygen, CH₂, NH, NR^{A} or hydrogen, in the case where Z is hydrogen then R₁ is not present, R^{A} is selected from the set defined for R₁ to R₅,
X is oxygen or NR^{A} providing that at least one X of General Formula I is NR^{A},
X may also combine independently with one of R₁ to R₅ to form an azide,
R₁ to R₅ are independently selected from the group comprising H, —(CO)R₆ or an alkyl, acyl, alkenyl, alkynyl, heteroalkyl, aryl, heteroaryl, arylalkyl or heteroarylalkyl substituent of 1 to 20 atoms, which is optionally substituted, and can be branched or linear wherein substituents include but are not limited to OH, NO, NO₂, NH₂, N₃, halogen, CF₃, CHF₂, CH₂F, nitrile, alkoxy, aryloxy, amidine, guanidiniums, carboxylic acid, carboxylic acid ester, carboxylic acid amide, aryl, cycloalkyl, heteroalkyl, heteroaryl, aminoalkyl, aminodialkyl, aminotrialkyl, aminoacyl, carbonyl, substituted or unsubstituted imine, sulfate, sulfonamide, phosphate, phosphoramide, hydrazide, hydroxamate, hydroxamic acid, heteroaryloxy, aminoalkyl, aminoaryl, aminoheteroaryl, thioalkyl, thioaryl or thioheteroaryl, which may optionally be further substituted, wherein R₆ is selected from the group comprising an alkyl, acyl, alkenyl, alkynyl, heteroalkyl, aryl, heteroaryl, arylalkyl or heteroarylalkyl substituent of 1 to 20 atoms, which is optionally substituted, and can be branched or linear wherein substituents include but are not limited to OH, NO, NO₂, NH₂, N₃, halogen, CF₃, CHF₂, CH₂F, nitrile, alkoxy, aryloxy, amidine, guanidiniums, carboxylic acid, carboxylic acid ester, carboxylic acid amide, aryl, cycloalkyl, heteroalkyl, heteroaryl, aminoalkyl, aminodialkyl, aminotrialkyl, aminoacyl, carbonyl, substituted or unsubstituted imine, sulfate, sulfonamide, phosphate, phosphoramide, hydrazide, hydroxamate, hydroxamic acid, heteroaryloxy, aminoalkyl, aminoaryl, aminoheteroaryl, thioalkyl, thioaryl or thioheteroaryl, which may optionally be further substituted,
with the proviso that XR₂, or XR₃ or XR₄ or XR₅ is not NH₂,
with the further proviso that not more than one of R₂ to R₅ is hydrogen,
where the group X is NR^{A} and R^{A} is not hydrogen, the groups R^{A} and the corresponding group R₂ to R₅ may combine to form a cycle.

In a preferred embodiment, the invention relates to the method wherein the compound is of general formula II

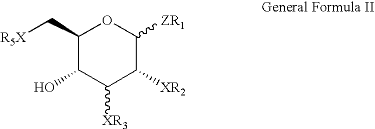

Wherein R₁, R₂, R₃, R₅, Z and X are defined as in General Formula I.

In a preferred embodiment, the invention relates to the method wherein the compound is of general formula III

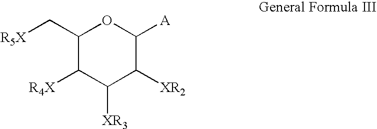

Wherein A is defined as hydrogen, SR₁, or OR₁ where R₁ is defined as in General Formula I, and

X and R₂ to R₅ are defined as in General Formula I.

In a preferred embodiment, the invention relates to the method wherein the compound is of General Formula IV

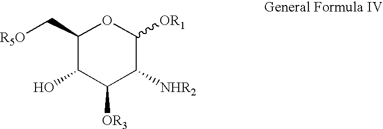

Wherein R₁-R₃ and R₅ are defined as in General Formula I.

In a preferred embodiment, the invention relates to the method wherein the compound is of General Formula V

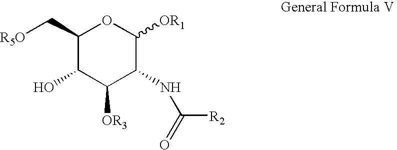

Wherein R₁-R₃ and R₅ are selected from the groups defined as in General Formula I, with the proviso that one of the groups R₁, R₂, R₃, or R₅ contains an acidic substituent including but not limited to: a carboxylate, a sulfonate, a phosfate, a hydroxamate, a phenol; or an adicic mimetic substituent including but not limited to: a tetrazole, an amide, an ester, a sulfonamide, a phosphoramide; and any of the remaining groups R₁, R₂, R₃, or R₅ contains a basic substituent including but not limited to: a primary amine, a secondary amine, a tertiary amine, a quaternary amine, an amidine, a guanidinium group, an imidazole group, a triazole group.

In a preferred embodiment, the invention relates to a compound according to any one of formula I, II, III, IV and V when used for treating a disease.

In a preferred embodiment, the invention relates to a compound according to any one of formula I, II, III, IV and V when used as a pharmaceutical.

In a preferred embodiment, the invention provides a method of treatment of a disease or condition affected by integrin inhibition which comprises administering an effective amount of a compound selected from the group consisting of formula I, II, III, IV or V, or a pharmaceutically acceptable salt thereof, to a subject in need.

In a preferred embodiment, the invention provides a method of treatment using a compound selected from the group consisting of formula I, II, III, IV or V, wherein the disease or condition is selected from the group consisting of diabetes, diabetic retinopathy, aged related macular degeneration, multiple sclerosis, asthma, arthritis, Crohn's disease and colitis, cancer, tumour metastasis, tumour growth, angiogenesis, neovascularisation, cardiovascular disorder, wound healing, thrombosis and osteoporosis, and related diseases or conditions.

In a preferred embodiment, the invention provides a compound when used according to the method wherein the compound is of Formula VI:

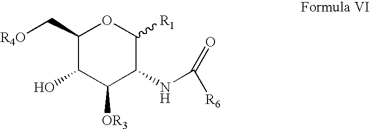

Wherein R₁ is selected from the group consisting of alkyl, hydroxy, alkoxy, aryloxy, arylalkyloxy, heteroaryloxy or benzyloxy; R₆ is alkyl, aryl, heteroaryl; R₃ is alkyl, aryl or arylalkyl; R₄ is aryl or arylalkyl; and wherein each of R₁, R₃, R₄ and R₆ may be further optionally substituted.

In a preferred embodiment, the invention provides a compound when used according to the method wherein R₁ is methoxy, ethoxy, hydroxyl, benzyloxy and phenoxy.

In a preferred embodiment, the invention provides a compound when used according to the method in which one of the groups R₁, R₃, R₄ or R₆ is substituted with a carboxylic acid or a carboxylic acid ester or a carboxylate anion or a carboxylate salt.

In a preferred embodiment, the invention provides a compound when used according to the method in which one of the groups R₃ or R₄ or R₆ is selected from the group consisting of hydroxy, methyl, ethyl, phenyl, benzyl, piperidine, triazole, tetrazole, imidazole, 4-aminomethylcyclohexane, carboxyphenyl, carboxybenzyl, chlorophenyl, bromobenzyl, amino phenyl, carboxymethylene, carboxyethylene, ethylguinidine, 4-guanidomethylphenyl, 3,5-diaminophenyl and (3,5-diaminophenyl)bis-formamide.

In a preferred embodiment, the invention provides a compound when used for treating diseases, wherein the compound is selected from the group consisting of:

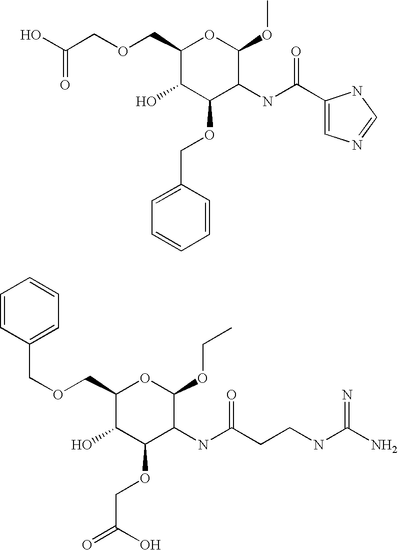

### DESCRIPTION OF THE INVENTION

The embodiments of the invention will be described with reference to the following examples. Where appropriate, the following abbreviations are used.
Ac Acetyl
DTPM 5-Acyl-1,3-dimethylbarbiturate
Ph Phenyl
TBDMS t-Butyldimethylsilyl
TBDPS t-Butyldiphenylsilyl
Bn benzyl
Bz benzoyl
Me methyl
DCE 1,2-dichloroethane
DCM dichloromethane, methylene chloride
Tf trifluoromethanesulfonyl
Ts 4-methylphenylsulfonyl, p-toluenesulfonyl
DMF N,N-dimethylformamide
DMAP N,N-dimethylaminopyridine
αα-DMT αα-dimethoxytoluene, benzaldehyde dimethyl acetal
DMSO dimethylsulfoxide
DTT dithiothreitol
DMTST Dimethyl(methylthio)sulphoniumtrifluoro-methanesulphonate
TBAF tetra-n-butylammonium fluoride

Compounds of the general structure were prepared according to methods disclosed in our earlier patent applications including PCT/AU03/001347, PCT/AU03/000384 and PCT/AU03/001008 the descriptions of which are incorporated by suitable cross reference. Exemplary methods of preparing compounds in solid and solution phase are provided herein.

### Part A: Preparation of Building Blocks

In order to fully enable the invention, we detail below methods for the preparation of certain building blocks used in the preparation of the compounds of the invention. The building blocks described are suitable for both solution and solid phase synthesis of the compounds of the invention.

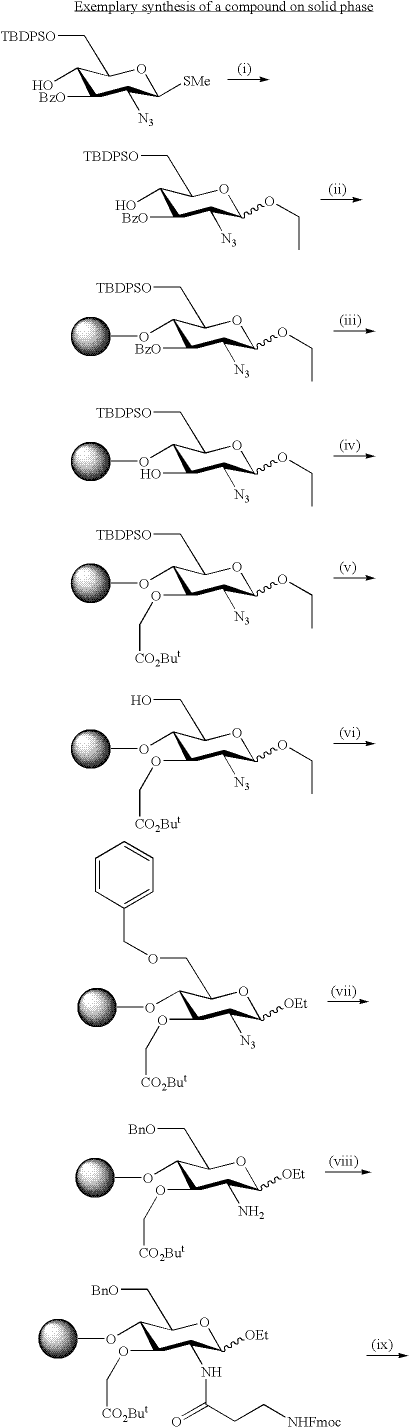

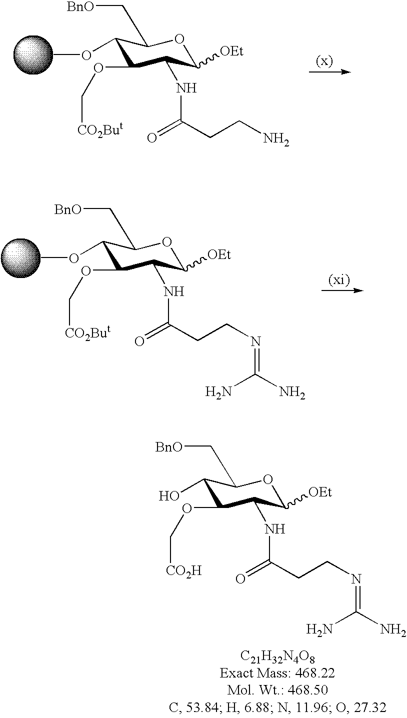

Conditions: (i) a. Br₂, DCM; b. Ethanol, silver triflate (AgOTf), DCM; (ii) TCA-Wang resin, boron trifluoride diethyl etherate (BF₃.Et₂O), DCM, tetrahydrofuran (THF); (iii) NaOMe, THF, MeOH; (iv) a. KOBu^{t}, DMF; b. t-Butyl-bromoglycolate, DMF; (v) HF.‘proton sponge’, acetic acid (AcOH), DMF, 65° C.; (vi) a. KOBu^{t}, DMF; b. Benzylbromide, DMF; (vii) 1,4-Dithio-DL-threitol, KOBu^{t}, DMF; (viii) HBTU, Fmoc-b-Ala-OH, di-isopropylethylamine (DIPEA), DMF; (ix) piperidine/DMF (1/4); (x) 3,5-dimethylpyrazolyl formamidinium nitrate, di-isopropylethylamine (DIPEA), DMF; (xi) TFA, Et₃SiH, DCM.

Further examples of compounds of the invention which may be prepared in solid phase include:

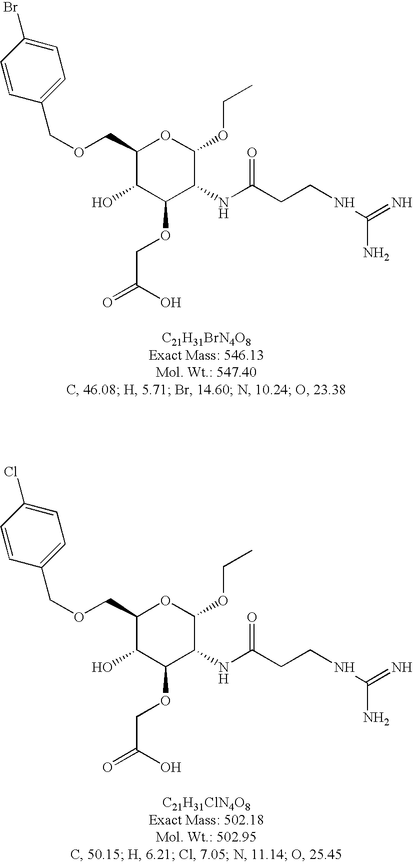

The bromobenzyl and chlorobenzyl compounds shown above are prepared according to conditions as listed above with bromobenzyl bromide and chlorobenzylbromide respectively used as alkylating agents in step (vi).

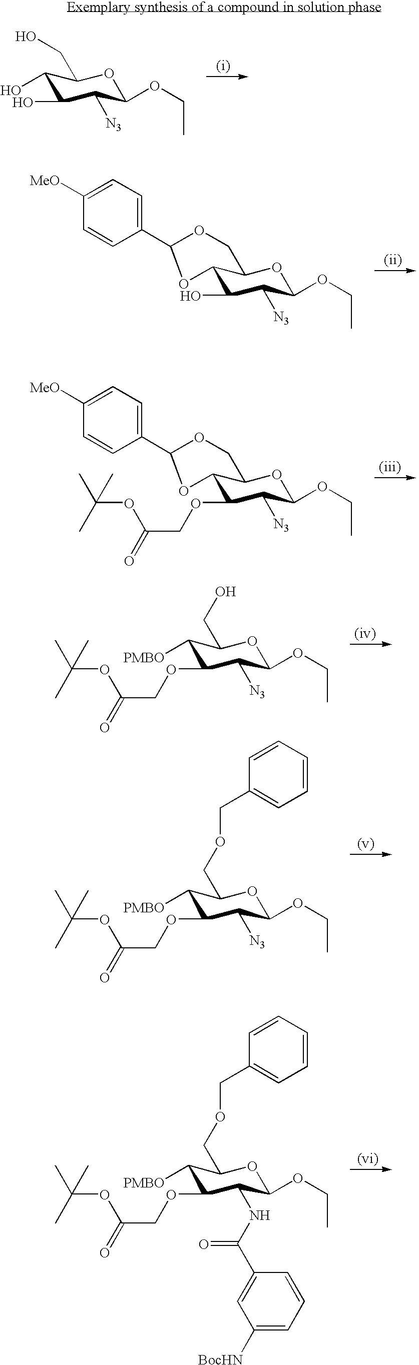

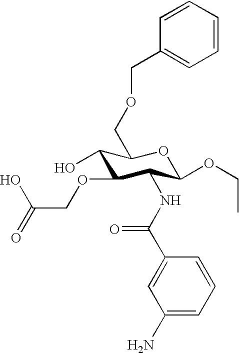

Conditions: (i) 4-Methoxybenzaldehyde dimethylacetal, p-toluenesulfonic acid (TsOH), CH₃CN; (ii) NaH (95%), tert-butyl bromoacetate, DMF; (iii) BH₃-THF, Bu₂BOTf, DCM; (iv) KOBu^{t}, BnBr, DMF; (v) a. Zn, NH₄Cl, MeOH, H₂O; b. 1-hydroxybenzotriazole-N,N,N′N′-tetramethyluronium hexafluorophosphate HBTU, 3-Boci-NH-benzoic acid, DIPEA, DMF; (vi) CH₃CN, H₂O, TsOH.

### Part B: Immobilization to Solid Support and Glycosylation:

The compounds of the present invention may be conveniently prepared in solution phase or on a solid support. Because a free hydroxyl group is always present in the compounds of the invention, it is convenient to immobilize the building blocks to the solid support through a hydroxy function which will become the free hydroxyl group in the final compounds. Many of the building blocks described above have a free hydroxyl in the 4 position which is suitable for immobilization. Where a free hydroxyl is desired in a different position, a protection/deprotection sequence is first performed.
Exemplary Immobilization onto Solid Phase

Wang resin (13.3 g; 0.85 mmol/g, p-Benzyloxybenzyl Alcohol polystyrene-divinylbenzene resin) was dried in the vacuum oven overnight in 500 ml round bottom flask. The flask was placed under nitrogen atmosphere then dry DCM (133 ml) and trichloroacetonitrile (20 ml) was added. The mixture was cooled with ice bath while gently stirred. After 15 minutes of cooling DBU (1.3 ml) was added drop wise in 15 minutes, the resulting mixture was stirred for one hour with ice bath cooling. The resin was collected by filtering, washed with DMF, THF and DCM (3× each). The resin was dried in the vacuum oven over P₂O₅ for 24 hours to afford 15 grams of TriChloroAcetimidate Wang (TCA-Wang) resin. The resin was packed under nitrogen and stored at 4° C.

Yield 100%; loading ca. 0.754 mmol/g.
(Alternative resins may be used).

Glycosylated building blocks containing one free hydroxyl are immobilised onto TCA-Wang resin. In a typical procedure, TCA Wang resin (3.6 gram) was dried in vacuum oven overnight then washed with anhydrous THF (3×36 ml) under nitrogen atmosphere. Building block (3 equiv.) was added followed by addition of anhydrous DCM (18 ml). The reaction mixture was shaken for 5 minutes (until all alcohol was dissolved), and BF₃.Et₂O (0.35 ml, 1 equivalent) was added. The reaction mixture was shaken vigorously for ten minutes and drained; the resin was washed with DCM (3×30 ml), DMF (3×30 ml), THF (3×30 ml) and dried.

### Part C: Library Preparation:

The compounds of the invention are prepared by sequential deprotection and ligation chemistries either on solid support or in solution phase. The following typical chemistries may be employed as required.

### Removal of a Tert-Butyldiphenylsilyl:

The resin bound building block is suspended in dry THF/methanol (20/1 v/v) mixture containing 10 equivalents of tetra-n-butylammonium fluoride. The mixture is stirred at 65° C. for 24 hours, drained; the resin is filtered, washed with dimethylformamide followed by THF and finally dichloromethane. In an alternative procedure, TBAF may be conveniently replaced by HF.pyridine and the reaction effected in plastic ware. The TBAF may also be replaced by HF.“proton sponge” complex with good results.

Removal of a Benzoate, p-Chlorobenzoate or Other Ester Protecting Group:

The resin bound building block is suspended in dry THF and methanol (3/1 v/v) mixture and sodium methoxide (0.5 equivalents) is added. The mixture is shaken for 24 hours, drained and re-treated with fresh reagents for further 24 hours. The resin is filtered, washed with dimethylformamide followed by THF and finally dichloromethane.

Removal of a p-Methoxybenzyl Group:

The resin bound building block is suspended in DCM and a small amount of water is added (approx 1%) followed by 2,3-dichloro-5,6-dicyanobenzoquinone (10 equivalents). The mixture is shaken for 3 hours, drained, and re-treated with fresh reagent for a further 3 hours. The resin is filtered, washed with THF followed by methanol and finally dichloromethane.

### Etherification of Hydroxyl Position:

Resin bound building block which has previously had a hydroxyl group deprotected is washed three times and then suspended in anhydrous DMF and 3 equivalents of potassium t-butoxide added (alternative bases may be employed), shaken and drained after 5 minutes followed by the alkylating agent (3 equivalents) in DMF. The mixture is shaken for 10 minutes, drained and re-treated twice more with fresh reagents as above. The resin is filtered, washed with dimethylformamide followed by THF and finally dichloromethane.

### Reduction of an Azide:

The resin bound building block is suspended in dry DMF; 5 equivalents of DTT (1,4-dithio-DL-threitol) and 3 equivalents of potassium tert-butoxide (alternative bases may be employed) are added. The mixture is agitated under nitrogen atmosphere for 24 hours, drained and the resin is washed with dimethylformamide followed by THF and finally dichloromethane.

### Removal of a DTPM Group:

The resin bound building block is suspended in DMF and hydrazine hydrate (50/1 v/v) mixture, agitated 2 hours, drained and the resin is washed with dimethylformamide followed by THF and finally dichloromethane.

### Amide Formation:

A solution of a suitable carboxylic acid (10 equivalents) in dry DMF is treated with HBTU (10 equivalents) and di-isopropylethylamine (10 equivalents) and shaken for 5 minutes. This solution is then added to a suspension of Resin bound building block, which has previously had an amine group deprotected in DMF and the mixture shaken for 30 minutes. After this time the resin is drained and treated once more with fresh reagent for 30 minutes. The resin is filtered, washed with DMF followed by methanol and finally dichloromethane. If desired, quantitative ninhydrin assay may be performed to determine that the reaction is complete. Alternative coupling systems including HOAT, EDC/NHS or anhydrides may be employed to similar effect.

### Removal of Fmoc:

The resin bound building block is suspended in piperidine/DMF (1/4, v/v) mixture and stirred 1 hours, drained and repeated once more; the resin is filtered, washed with dimethylformamide followed by THF and finally dichloromethane.

### Guanidine Formation:

The resin bound building block is suspended in dry DMF containing 3 equivalents of 3,5-dimethylpyrazolyl formamidinium nitrate and 15 equivalents of DIPEA. The mixture is stirred at 65° C. for 24 hours, drained; the resin is filtered, washed with dimethylformamide followed by THF and finally dichloromethane.

### Cleavage of Resin Bound Product:

The resin bound compound is suspended in dry DCM containing 20% TFA and 20% Et₃SiH. The mixture is stirred at RT for 3 hours and the aliquot was collected; the resin was washed with dry DCM and all the DCM solutions were combined, evaporated to dryness under reduced vacuo to furnish the desired product.

The compounds were tested against 2 integrins and the relative inhibition is presented in the following table. Inhibition is designated according to the following categories: 0% to 35% inhibition at 250 micromolar=“−”; 36% to 60% inhibition at 250 micromolar=“+”; 61% to 80% inhibition at 250 micromolar=“++”; 81% to 100% inhibition at 250 micromolar=“+++”,

### Biological Assay:

An ELISA assay based on the published method of Bethert et al., 2000, J Biol Chem 275, 33308-23, was employed.

Briefly, appropriate microtitre plates were coated with either Fibrinogen or Vitronectin (10 μg/well). These extracellular matrix proteins contain the RGD amino acid sequence that is recognized by α_{llb}β₃ integrin. Human platelet membrane preparations were used as a source of α_{llb}β₃ integrin and the cell line WM-115 was used as a source of α_{V}β₃ integrin. Inhibition of the binding of α_{llb}β₃ integrin containing membrane preparations to the extracellular matrix protein was determined by pre-incubating the platelet membrane preparation with test or control compounds. The binding of the α_{llb}β₃ integrin containing membrane was the quantitated by using a rabbit anti-integrinβ3 antibody, a horse radish peroxidase coupled second antibody and a standard colorimetric detection system.

Compounds tested are indicated in Table 1 below, and are of the general formula:

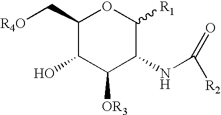

NOTE: Individual isomers were separated and tested as separate entities.

TABLE 1
Intergrin Binding Activity of compounds

| Compound | | | | |
|---|---|---|---|---|
| Number | R1 | R2 | R3 | R4 |
| 1 | OH | -(3-aminophenyl) | -(4-bromobenzyl) | —CH2-CO2H |
| 2 | OH | -(3-aminophenyl) | -(4-bromobenzyl) | —CH2-CO2H |
| 3 | OMe | -(4-carboxyphenyl) | -(4-bromobenzyl) | -(3-aminobenzyl) |
| 4 | OMe | -(4-carboxyphenyl) | -(4-bromobenzyl) | -(3-aminobenzyl) |
| 5 | OMe | -(3-aminophenyl) | —CH2-CO2H | -Bn |
| 6 | OMe | -(3-aminophenyl) | —CH2-CO2H | -Bn |
| 7 | OMe | -(3-aminophenyl) | —CH2-CO2H | -(4-bromobenzyl) |
| 8 | OMe | -(3-aminophenyl) | —CH2-CO2H | -(4-bromobenzyl) |
| 9 | OMe | -(4-chlorophenyl) | —CH2-CO2H | -(3-aminobenzyl) |
| 10 | OMe | -(4-chlorophenyl) | —CH2-CO2H | -(3-aminobenzyl) |
| 11 | OMe | —CH2-CH2-NH—C— | -(4-carboxybenzyl) | -Bn |
| | | (═NH)—NH2 | | |
| 12 | OMe | —CH2-CH2-NH—C— | -(4-carboxybenzyl) | -Bn |
| | | (═NH)—NH2 | | |
| 13 | OMe | —CH2-CH2-NH—C— | -(4-carboxybenzyl) | -Bn |
| | | (═NH)—NH2 | methyl ester | |
| 14 | OMe | —CH2-CH2-NH—C— | -(4-carboxybenzyl) | -Bn |
| | | (═NH)—NH2 | methyl ester | |
| 15 | OMe | —CH2-CH2-NH—C— | -(4-carboxybenzyl) | -(4-bromobenzyl) |
| | | (═NH)—NH2 | | |
| 16 | OMe | —CH2-CH2-NH—C— | -(4-carboxybenzyl) | -(4-bromobenzyl) |
| | | (═NH)—NH2 | | |
| 17 | OMe | —CH2-CH2-NH—C— | -(4-carboxybenzyl) | -(4-bromobenzyl) |
| | | (═NH)—NH2 | methyl ester | |
| 18 | OMe | -phenyl | -(4-carboxybenzyl) | -(3-aminobenzyl) |
| 19 | OMe | -phenyl | -(4-carboxybenzyl) | -(3-aminobenzyl) |
| 20 | OMe | —CH2-CH2-CO2H | -(3-aminobenzyl) | -Bn |
| 21 | OMe | —CH2-CH2-CO2H | -(3-aminobenzyl) | -Bn |
| 22 | OH | —CH2-CH2-CO2H | -(3-aminobenzyl) | -Bn |
| 23 | OH | —CH2-CH2-CO2H | -(3-aminobenzyl) | -Bn |
| 24 | OMe | —CH2-CH2-CO2H | -(3-aminobenzyl) | -(4-bromobenzyl) |
| 25 | OMe | —CH2-CH2-CO2H | -(3-aminobenzyl) | -(4-bromobenzyl) |
| 26 | OH | —CH2-CH2-CO2H | -(3-aminobenzyl) | -(4-bromobenzyl) |
| 27 | OMe | -phenyl | -(3-aminobenzyl) | —CH2-CO2H |
| 28 | OMe | -phenyl | -(3-aminobenzyl) | —CH2-CO2H |
| 29 | OH | -phenyl | -(3-aminobenzyl) | —CH2-CO2H |
| 30 | OMe | -(4-chlorophenyl) | -(3-aminobenzyl) | -(4-carboxybenzyl) |
| 31 | OMe | -(4-chlorophenyl) | -(3-aminobenzyl) | -(4-carboxybenzyl) |
| 32 | OEt | -(3-aminophenyl) | -Bn | —CH2-CO2H |
| 33 | OH | -(3-aminophenyl) | -Bn | —CH2-CO2H |
| 34 | OH | -(3-aminophenyl) | -Bn | —CH2-CO2H |
| 35 | OEt | -(3-aminophenyl) | -Bn | -(4-carboxybenzyl) |
| 36 | OEt | -(4-carboxyphenyl) | -Bn | -(3-aminobenzyl) |
| 37 | OEt | -(4-carboxyphenyl) | -Bn | -(3-aminobenzyl) |
| 38 | OEt | -(3-aminophenyl) | -(4-bromobenzyl) | —CH2-CO2H |
| 39 | OEt | -(3-aminophenyl) | -(4-bromobenzyl) | —CH2-CO2H |
| 40 | OEt | -(3-aminophenyl) | -(4-bromobenzyl) | -(4-carboxybenzyl) |
| 41 | OEt | -(3-aminophenyl) | -(4-bromobenzyl) | -(4-carboxybenzyl) |
| 42 | OH | -(3-aminophenyl) | -(4-bromobenzyl) | -(4-carboxybenzyl) |
| 43 | OH | -(3-aminophenyl) | -(4-bromobenzyl) | -(4-carboxybenzyl) |
| 44 | OEt | —CH2-CH2-CO2H | -(4-bromobenzyl) | -(3-aminobenzyl) |
| 45 | OEt | —CH2-CH2-CO2H | -(4-bromobenzyl) | -(3-aminobenzyl) |
| 46 | OH | —CH2-CH2-CO2H | -(4-bromobenzyl) | -(3-aminobenzyl) |
| 47 | OH | —CH2-CH2-CO2H | -(4-bromobenzyl) | -(3-aminobenzyl) |
| 48 | OEt | —CH2-CH2-NH—C— | —CH2-CO2H | -Bn |
| | | (═NH)—NH2 | | |
| 49 | OEt | —CH2-CH2-NH—C— | —CH2-CO2H | -Bn |
| | | (═NH)—NH2 | | |
| 50 | OEt | —CH2-CH2-NH—C— | —CH2-CO2H | -(4-bromobenzyl) |
| | | (═NH)—NH2 | | |
| 51 | OEt | —CH2-CH2-NH—C— | —CH2-CO2H | -(4-bromobenzyl) |
| | | (═NH)—NH2 | | |
| 52 | OEt | —CH2-CH2-NH—C— | —CH2-CO2H | -(4-bromobenzyl) |
| | | (═NH)—NH2 | | |
| 53 | OEt | —CH2-CH2-NH—C— | —CH2-CO2H | -(4-bromobenzyl) |
| | | (═NH)—NH2 | | |
| 54 | OEt | -phenyl | —CH2-CO2H | -(3-aminobenzyl) |
| 55 | OEt | -phenyl | —CH2-CO2H | -(3-aminobenzyl) |
| 56 | H | -phenyl | —CH2-CO2H | -(3-aminobenzyl) |
| 57 | OEt | —CH2-CH2-NH—C— | -(4-carboxybenzyl) | -Bn |
| | | (═NH)—NH2 | | |
| 58 | OEt | —CH2-CH2-NH—C— | -(4-carboxybenzyl) | -Bn |
| | | (═NH)—NH2 | | |
| 59 | OH | —CH2-CH2-NH—C— | -(4-carboxybenzyl) | -Bn |
| | | (═NH)—NH2 | | |
| 60 | OEt | —CH2-CH2-NH—C— | -(4-carboxybenzyl) | -(4-bromobenzyl) |
| | | (═NH)—NH2 | | |
| 61 | OEt | —CH2-CH2-NH—C— | -(4-carboxybenzyl) | -(4-bromobenzyl) |
| | | (═NH)—NH2 | | |
| 62 | OEt | —CH2-CH2-NH—C— | -(4-carboxybenzyl) | -(4-bromobenzyl) |
| | | (═NH)—NH2 | | |
| 63 | OH | —CH2-CH2-NH—C— | -(4-carboxybenzyl) | -(4-bromobenzyl) |
| | | (═NH)—NH2 | | |
| 64 | OEt | -(4-chlorophenyl) | -(4-carboxybenzyl) | -(3-aminobenzyl) |
| 65 | OEt | -(4-chlorophenyl) | -(4-carboxybenzyl) | -(3-aminobenzyl) |
| 66 | OH | -(4-chlorophenyl) | -(4-carboxybenzyl) | -(3-aminobenzyl) |
| 67 | OH | -(4-chlorophenyl) | -(4-carboxybenzyl) | -(3-aminobenzyl) |
| 68 | OEt | -(4-carboxyphenyl) | -(3-aminobenzyl) | -Bn |
| 69 | OEt | -(4-carboxyphenyl) | -(3-aminobenzyl) | -Bn |
| 70 | OH | -(4-carboxyphenyl) | -(3-aminobenzyl) | -Bn |
| 71 | OH | -(4-carboxyphenyl) | -(3-aminobenzyl) | -Bn |
| 72 | OEt | -(4-carboxyphenyl) | -(3-aminobenzyl) | -(4-bromobenzyl) |
| 73 | OEt | -(4-carboxyphenyl) | -(3-aminobenzyl) | -(4-bromobenzyl) |
| 74 | OH | -(4-carboxyphenyl) | -(3-aminobenzyl) | -(4-bromobenzyl) |
| 75 | OH | -(4-carboxyphenyl) | -(3-aminobenzyl) | -(4-bromobenzyl) |
| 76 | OEt | -(4-chlorophenyl) | -(3-aminobenzyl) | —CH2-CO2H |
| 77 | OEt | -(4-chlorophenyl) | -(3-aminobenzyl) | —CH2-CO2H |
| 78 | OEt | -(4-chlorophenyl) | -(3-aminobenzyl) | -(4-carboxybenzyl) |
| 79 | OEt | -(4-chlorophenyl) | -(3-aminobenzyl) | -(4-carboxybenzyl) |
| 80 | H | -(4-chlorophenyl) | -(3-aminobenzyl) | —CH2-CO2H |
| 81 | OH | -(4-chlorophenyl) | -(3-aminobenzyl) | -(4-carboxybenzyl) |
| 82 | OH | -(4-chlorophenyl) | -(3-aminobenzyl) | -(4-carboxybenzyl) |
| 83 | OH | -(4-chlorophenyl) | -(3-aminobenzyl) | -(4-carboxybenzyl) |
| 84 | OBn | -(3-aminophenyl) | -Et | —CH2-CO2H |
| 85 | OBn | -(3-aminophenyl) | -Et | —CH2-CO2H |
| 86 | OBn | —CH2-CH2-NH—C— | -Et | -(4-carboxybenzyl) |
| | | (═NH)—NH2 | | |
| 87 | OBn | —CH2-CH2-NH—C— | -Et | -(4-carboxybenzyl) |
| | | (═NH)—NH2 | | |
| 88 | OBn | —CH2-CH2-CO2H | -Et | -(3-aminobenzyl) |
| 89 | OBn | —CH2-CH2-CO2H | -Et | -(3-aminobenzyl) |
| 90 | OBn | —CH2-CH2-NH—C— | —CH2-CO2H | -Me |
| | | (═NH)—NH2 | | |
| 91 | OBn | —CH2-CH2-NH—C— | —CH2-CO2H | -Me |
| | | (═NH)—NH2 | | |
| 92 | OBn | —CH2-CH2-NH—C— | —CH2-CO2H | -Et |
| | | (═NH)—NH2 | | |
| 93 | OBn | —CH2-CH2-NH—C— | —CH2-CO2H | -Et |
| | | (═NH)—NH2 | | |
| 94 | OBn | —CH2-CH2-NH—C— | —CH2-CO2H | H |
| | | (═NH)—NH2 | | |
| 95 | OBn | -Me | —CH2-CO2H | -(3-aminobenzyl) |
| 96 | OBn | -Me | —CH2-CO2H | -(3-aminobenzyl) |
| 97 | OBn | —CH2-CH2-NH—C— | -(4-carboxybenzyl) | -Me |
| | | (═NH)—NH2 | | |
| 98 | OBn | —CH2-CH2-NH—C— | -(4-carboxybenzyl) | -Me |
| | | (═NH)—NH2 | | |
| 99 | OBn | -(3-aminophenyl) | -(4-carboxybenzyl) | -Et |
| 100 | OBn | -(3-aminophenyl) | -(4-carboxybenzyl) | -Et |
| 101 | OBn | -(3-aminophenyl) | -(4-carboxybenzyl) | H |
| 102 | OBn | -(3-aminophenyl) | -(4-carboxybenzyl) | H |
| 103 | OBn | -Et | -(4-carboxybenzyl) | -(3-aminobenzyl) |
| 104 | OBn | -Et | -(4-carboxybenzyl) | -(3-aminobenzyl) |
| 105 | OBn | -(4-carboxyphenyl) | -(3-aminobenzyl) | -Me |
| 106 | OBn | -(4-carboxyphenyl) | -(3-aminobenzyl) | -Me |
| 107 | OBn | -(4-carboxyphenyl) | -(3-aminobenzyl) | -Et |
| 108 | OBn | -(4-carboxyphenyl) | -(3-aminobenzyl) | -Et |
| 109 | OBn | -(4-carboxyphenyl) | -(3-aminobenzyl) | H |
| 110 | OBn | -Et | -(3-aminobenzyl) | —CH2-CO2H |
| 111 | OBn | -Et | -(3-aminobenzyl) | -(4-carboxybenzyl) |
| 112 | OBn | -Et | -(3-aminobenzyl) | -(4-carboxybenzyl) |
| 113 | O—CH2-CH2-NH— | -Me | -Bn | —CH2-CO2H |
| | C(═NH)—NH2 | | | |
| 114 | O—CH2-CH2-NH— | -phenyl | -(4-carboxybenzyl) | -Me |
| | C(═NH)—NH2 | | | |
| 115 | O—CH2-CH2-NH— | -phenyl | -(4-carboxybenzyl) | -Me |
| | C(═NH)—NH2 | | | |
| 116 | OEt | —CH2-NH2 | -Bn | —CH2-CO2H |
| 117 | OEt | —CH2-NH2 | -Bn | —CH2-CO2H |
| 118 | OEt | —CH2-NH—C—(═NH)—NH2 | -Bn | —CH2-CO2H |
| 119 | OEt | —CH2-NH—C—(═NH)—NH2 | -Bn | —CH2-CO2H |
| 120 | OEt | —CH2-CH2-NH2 | -Bn | —CH2-CO2H |
| 121 | OEt | —CH2-CH2-NH2 | -Bn | —CH2-CO2H |
| 122 | OEt | —CH2-CH2-NH—C— | -Bn | —CH2-CO2H |
| | | (═NH)—NH2 | | |
| 123 | OMe | —CH2-CH2-CH2-NH2 | -Bn | —CH2-CO2H |
| 124 | OMe | —CH2-CH2-CH2-NH2 | -Bn | —CH2-CO2H |
| 125 | OMe | —CH2-CH2-CH2-NH—C— | -Bn | —CH2-CO2H |
| | | (═NH)—NH2 | | |
| 126 | OMe | —CH2-CH2-CH2-NH—C— | -Bn | —CH2-CO2-CH3 |
| | | (═NH)-NH2 | | |
| 127 | OMe | -(4-aminomethyl)phenyl | -Bn | —CH2-CO2H |
| 128 | OMe | -(4-guanadinomethyl)phenyl | -Bn | —CH2-CO2H |
| 129 | OMe | -(4-guanadinomethyl)phenyl | -Bn | —CH2-CO2H |
| 130 | OMe | -(3,5-diaminophenyl) | -Bn | —CH2-CO2H |
| 131 | OMe | -(3,5-diaminophenyl) | -Bn | —CH2-CO2H |
| 132 | OMe | -3′-imidazole | -Bn | —CH2-CO2H |
| 133 | OMe | -3′-imidazole | -Bn | —CH2-CO2H |
| 134 | OMe | -4′-piperidine | -Bn | —CH2-CO2H |
| 135 | OMe | -4′-piperidine | -Bn | —CH2-CO2H |
| 136 | OMe | -4′-pipeiridine | -Bn | —CH2-CO2H |
| 137 | OMe | -4-aminomethylcyclohexane | -Bn | —CH2-CO2H |
| 138 | OMe | -4-aminomethylcyclohexane | -Bn | —CH2-CO2H |
| 139 | H | -(4-carboxyphenyl) | -(4-bromobenzyl) | —CH2-CO2H |
| 140 | H | -(4-carboxyphenyl ) | -(3-aminobenzyl) | -Bn |
| 141 | H | -(4-chlorophenyl) | -(3-aminobenzyl) | -(4-carboxybenzyl) |
| 142 | H | -(3-aminophenyl) | -Et | —CH2-CO2H |
| 143 | H | —CH2-CH2-NH—C— | -Et | -(4-carboxybenzyl) |
| | | (═NH)—NH2 | | |
| 144 | H | -(4-guanadinomethyl)phenyl | -Bn | —CH2-CO2H |
| 145 | H | -(3,5-diaminophenyl) | -Bn | —CH2-CO2H |
| | | bis formamide | | |

| | INHIBITION @ 250 uM | INHIBITION 250 uM | INHIBITION 250 uM |
|---|---|---|---|
| | RECEPTOR allBb3 | RECEPTOR allBb3 | RECEPTOR aVb3 |
| Compound | SUBSTRATE | SUBSTRATE | SUBSTRATE |
| Number | FIBRINOGEN | VITRONECTIN | FIBRINOGEN |
| 1 | + | + | − |
| 2 | ++ | +++ | ++ |
| 3 | + | +++ | + |
| 4 | − | − | + |
| 5 | ++ | +++ | ++ |
| 6 | − | +++ | +++ |
| 7 | + | +++ | + |
| 8 | + | +++ | +++ |
| 9 | + | +++ | +++ |
| 10 | ++ | +++ | +++ |
| 11 | + | +++ | + |
| 12 | ++ | ++ | − |
| 13 | + | +++ | − |
| 14 | + | +++ | ++ |
| 15 | + | +++ | ++ |
| 16 | + | +++ | ++ |
| 17 | + | +++ | +++ |
| 18 | ++ | +++ | +++ |
| 19 | ++ | +++ | +++ |
| 20 | +++ | +++ | +++ |
| 21 | +++ | +++ | +++ |
| 22 | + | + | + |
| 23 | ++ | +++ | − |
| 24 | + | +++ | ++ |
| 25 | + | +++ | +++ |
| 26 | + | +++ | ++ |
| 27 | ++ | +++ | +++ |
| 28 | + | +++ | +++ |
| 29 | ++ | +++ | +++ |
| 30 | ++ | +++ | +++ |
| 31 | +++ | +++ | +++ |
| 32 | + | +++ | − |
| 33 | ++ | − | − |
| 34 | + | +++ | +++ |
| 35 | + | +++ | +++ |
| 36 | + | +++ | +++ |
| 37 | + | +++ | +++ |
| 38 | ++ | +++ | +++ |
| 39 | + | +++ | +++ |
| 40 | +++ | +++ | +++ |
| 41 | + | ++ | + |
| 42 | + | +++ | − |
| 43 | n.d. | n.d. | n.d. |
| 44 | ++ | − | + |
| 45 | ++ | +++ | +++ |
| 46 | + | +++ | +++ |
| 47 | + | +++ | − |
| 48 | ++ | +++ | +++ |
| 49 | ++ | +++ | +++ |
| 50 | + | +++ | +++ |
| 51 | ++ | +++ | ++ |
| 52 | + | ++ | +++ |
| 53 | n.d. | n.d. | n.d. |
| 54 | − | ++ | + |
| 55 | + | +++ | +++ |
| 56 | − | +++ | ++ |
| 57 | − | +++ | +++ |
| 58 | + | +++ | +++ |
| 59 | + | +++ | +++ |
| 60 | + | +++ | +++ |
| 61 | − | +++ | +++ |
| 62 | n.d. | n.d. | n.d. |
| 63 | ++ | +++ | − |
| 64 | − | ++ | + |
| 65 | − | +++ | − |
| 66 | + | +++ | + |
| 67 | + | +++ | − |
| 68 | − | ++ | ++ |
| 69 | − | +++ | +++ |
| 70 | + | +++ | +++ |
| 71 | − | +++ | +++ |
| 72 | + | +++ | +++ |
| 73 | ++ | +++ | − |
| 74 | − | +++ | − |
| 75 | + | +++ | − |
| 76 | − | − | − |
| 77 | ++ | +++ | + |
| 78 | − | +++ | +++ |
| 79 | + | +++ | +++ |
| 80 | ++ | +++ | + |
| 81 | + | +++ | +++ |
| 82 | ++ | + | ++ |
| 83 | − | +++ | +++ |
| 84 | − | − | − |
| 85 | − | +++ | − |
| 86 | − | +++ | − |
| 87 | − | +++ | − |
| 88 | − | ++ | − |
| 89 | − | +++ | + |
| 90 | − | +++ | − |
| 91 | + | + | − |
| 92 | + | +++ | ++ |
| 93 | − | +++ | +++ |
| 94 | − | + | − |
| 95 | − | ++ | − |
| 96 | − | + | − |
| 97 | − | − | − |
| 98 | − | +++ | − |
| 99 | − | ++ | − |
| 100 | − | +++ | − |
| 101 | − | ++ | + |
| 102 | − | +++ | +++ |
| 103 | ++ | +++ | +++ |
| 104 | − | +++ | − |
| 105 | +++ | ++ | − |
| 106 | + | +++ | − |
| 107 | − | +++ | − |
| 108 | − | +++ | − |
| 109 | − | +++ | − |
| 110 | − | +++ | − |
| 111 | − | +++ | − |
| 112 | − | +++ | +++ |
| 113 | + | +++ | +++ |
| 114 | − | ++ | − |
| 115 | − | − | − |
| 116 | − | − | − |
| 117 | + | +++ | − |
| 118 | − | +++ | − |
| 119 | − | +++ | − |
| 120 | + | + | − |
| 121 | n.d. | n.d. | n.d. |
| 122 | ++ | ++ | − |
| 123 | + | +++ | − |
| 124 | ++ | +++ | + |
| 125 | + | +++ | + |
| 126 | ++ | +++ | +++ |
| 127 | − | − | − |
| 128 | − | +++ | − |
| 129 | − | +++ | − |
| 130 | − | +++ | − |
| 131 | + | +++ | − |
| 132 | + | ++ | − |
| 133 | ++ | +++ | − |
| 134 | +++ | +++ | − |
| 135 | n.d. | n.d. | n.d. |
| 136 | +++ | +++ | − |
| 137 | +++ | +++ | ++ |
| 138 | − | +++ | − |
| 139 | n.d. | n.d. | n.d. |
| 140 | n.d. | n.d. | n.d. |
| 141 | n.d. | n.d. | n.d. |
| 142 | n.d. | n.d. | n.d. |
| 143 | n.d. | n.d. | n.d. |
| 144 | n.d. | n.d. | n.d. |
| 145 | n.d. | n.d. | n.d. |

### REFERENCES

1. Edwin A. Clark and Joan S. Brugge, Science, 1995, 268, 233-239.
2. M. Amin Arnout, Simon L. Goodman and Jian-Ping Xiong, Current Opinion in Cell Biology, 2002, 14, 641-651

Throughout the specification and the claims (if present), unless the context requires otherwise, the term “comprise”, or variations such as “comprises” or “comprising”, will be understood to apply the inclusion of the stated integer or group of integers but not the exclusion of any other integer or group of integers.

Throughout the specification and claims (if present), unless the context requires otherwise, the term “substantially” or “about” will be understood to not be limited to the value for the range qualified by the terms.

It should be appreciated that various other changes and modifications can be made to any embodiment described without departing from the spirit and scope of the invention.

1. A method of inhibiting or effecting the activity of an integrin receptor which comprises contacting an integrin with an effective amount of a compound of formula I, or a pharmaceutically acceptable salt thereof; 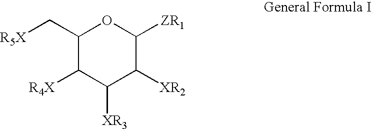
wherein the ring may be of any configuration;
Z is sulphur, oxygen, CH₂, NH, NR^{A} or hydrogen, in the case where Z is hydrogen then R₁ is not present, R^{A} is selected from the set defined for R₁ to R₅,
X is oxygen or NR^{A} providing that at least one X of General Formula I is NR^{A}, X may also combine independently with one of R₁ to R₅ to form an azide,
R₁ to R₅ are independently selected from the group comprising H, —(CO)R₆ or an alkyl, acyl, alkenyl, alkynyl, heteroalkyl, aryl, heteroaryl, arylalkyl or heteroarylalkyl substituent of 1 to 20 atoms, which is optionally substituted, and can be branched or linear wherein substituents are selected from the group consisting of: OH, NO, NO₂, NH₂, N₃, halogen, CF₃, CHF₂, CH₂F, nitrile, alkoxy, aryloxy, amidine, guanidiniums, carboxylic acid, carboxylic acid ester, carboxylic acid amide, aryl, cycloalkyl, heteroalkyl, heteroaryl, aminoalkyl, aminodialkyl, aminotrialkyl, aminoacyl, carbonyl, substituted or unsubstituted imine, sulfate, sulfonamide, phosphate, phosphoramide, hydrazide, hydroxamate, hydroxamic acid, heteroaryloxy, aminoalkyl, aminoaryl, aminoheteroaryl, thioalkyl, thioaryl or thioheteroaryl, which may optionally be further substituted, wherein R₆ is selected from the group comprising an alkyl, acyl, alkenyl, alkynyl, heteroalkyl, aryl, heteroaryl, arylalkyl or heteroarylalkyl substituent of 1 to 20 atoms, which is optionally substituted, and can be branched or linear wherein substituents include but are not limited to OH, NO, NO₂, NH₂, N₃, halogen, CF₃, CHF₂, CH₂F, nitrile, alkoxy, aryloxy, amidine, guanidiniums, carboxylic acid, carboxylic acid ester, carboxylic acid amide, aryl, cycloalkyl, heteroalkyl, heteroaryl, aminoalkyl, aminodialkyl, aminotrialkyl, aminoacyl, carbonyl, substituted or unsubstituted imine, sulfate, sulfonamide, phosphate, phosphoramide, hydrazide, hydroxamate, hydroxamic acid, heteroaryloxy, aminoalkyl, aminoaryl, aminoheteroaryl, thioalkyl, thioaryl or thioheteroaryl, which may optionally be further substituted,
with the proviso that XR₂, or XR₃ or XR₄ or XR₅ is not NH₂,
with the further proviso that not more than one of R₂ to R₅ is hydrogen,
where the group X is NR^{A} and R^{A} is not hydrogen, the groups R^{A} and the corresponding group R₂ to R₅ may combine to form a cycle.

2. The method of claim 1 wherein the compound is of general formula II: 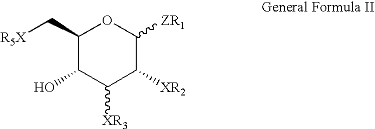
or a pharmaceutically acceptable salt thereof;
wherein R₁, R₂, R₃, R₅, Z and X are defined as in General Formula I.

3. The method of claim 1 wherein the compound is of general formula III: 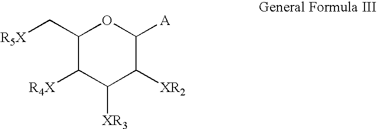
or a pharmaceutically acceptable salt thereof;
wherein A is defined as hydrogen, SR₁, or OR₁ where R₁ is defined as in General Formula I, and
X and R₂ to R₅ are defined as in General Formula I.

4. The method of claim 1 wherein the compound is of General Formula IV: 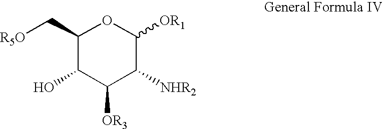
or a pharmaceutically acceptable salt thereof;
wherein R₁-R₃ and R₅ are defined as in General Formula I.

5. The method of claim 1 wherein the compound is of General Formula V: 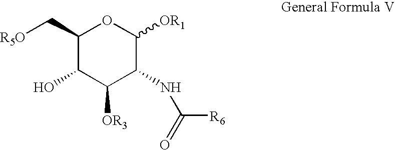
or a pharmaceutically acceptable salt thereof;
wherein R₁, R₃, R₅ and R₆ are independently selected from the group comprising an alkyl, alkenyl, alkynyl, heteroalkyl, aryl, heteroaryl, arylalkyl or heteroarylalkyl substituent of 1 to 20 atoms, which is optionally substituted, and can be branched or linear wherein substituents include but are not limited to OH, NO, NO₂, NH₂, N₃, halogen, CF₃, CHF₂, CH₂F, nitrile, alkoxy, aryloxy, amidine, guanidiniums, carboxylic acid, carboxylic acid ester, carboxylic acid amide, aryl, cycloalkyl, heteroalkyl, heteroaryl, aminoalkyl, aminodialkyl, aminotrialkyl, aminoacyl, carbonyl, substituted or unsubstituted imine, sulfate, sulfonate, sulfonamide, phosphate, phosphoramide, hydrazide, hydroxamate, hydroxamic acid, heteroaryloxy, aminoalkyl, aminoaryl, aminoheteroaryl, thioalkyl, thioaryl or thioheteroaryl, which may optionally be further substituted, with the proviso that one of the groups R₁, R₃, R₅, or R₆ contains an acidic substituent including but not limited to: a carboxylate, a sulfonate, a phosphate, a hydroxamate, a phenol; or an adic mimetic substituent including but not limited to: a tetrazole, an amide, an ester, a sulfonamide, a phosphoramide; and any of the remaining groups R₁, R₃, R₅, or R₆ contains a basic substituent including but not limited to: a primary amine, a secondary amine, a tertiary amine, a quaternary amine, an amidine, a guanidinium group, an imidazole group, a triazole group.

6-7. (canceled)

8. A method of treatment of a disease or condition affected by integrin inhibition which comprises administering an effective amount of a compound of formula I, 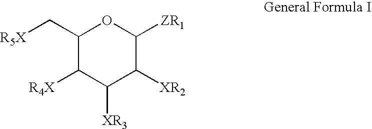
wherein the ring may be of any configuration;
Z is sulphur, oxygen, CH₂, NH, NR^{A} or hydrogen, in the case where Z is hydrogen then R₁ is not present, R^{A} is selected from the set defined for R₁ to R₅,
X is oxygen or NR^{A} providing that at least one X of General Formula I is NR^{A}, X may also combine independently with one of R₁ to R₅ to form an azide,
R₁ to R₅ are independently selected from the group comprising H, —(CO)R₆ or an alkyl, acyl, alkenyl, alkynyl, heteroalkyl, aryl, heteroaryl, arylalkyl or heteroarylalkyl substituent of 1 to 20 atoms, which is optionally substituted, and can be branched or linear wherein substituents are selected from the group consisting of: OH, NO, NO₂, NH₂, N₃, halogen, CF₃, CHF₂, CH₂F, nitrile, alkoxy, aryloxy, amidine, guanidiniums, carboxylic acid, carboxylic acid ester, carboxylic acid amide, aryl, cycloalkyl, heteroalkyl, heteroaryl, aminoalkyl, aminodialkyl, aminotrialkyl, aminoacyl, carbonyl, substituted or unsubstituted imine, sulfate, sulfonamide, phosphate, phosphoramide, hydrazide, hydroxamate, hydroxamic acid, heteroaryloxy, aminoalkyl, aminoaryl, aminoheteroaryl, thioalkyl, thioaryl or thioheteroaryl, which may optionally be further substituted, wherein R₆ is selected from the group comprising an alkyl, acyl, alkenyl, alkynyl, heteroalkyl, aryl, heteroaryl, arylalkyl or heteroarylalkyl substituent of 1 to 20 atoms, which is optionally substituted, and can be branched or linear wherein substituents include but are not limited to OH, NO, NO₂, NH₂, N₃, halogen, CF₃, CHF₂, CH₂F, nitrile, alkoxy, aryloxy, amidine, guanidiniums, carboxylic acid, carboxylic acid ester, carboxylic acid amide, aryl, cycloalkyl, heteroalkyl, heteroaryl, aminoalkyl, aminodialkyl, aminotrialkyl, aminoacyl, carbonyl, substituted or unsubstituted imine, sulfate, sulfonamide, phosphate, phosphoramide, hydrazide, hydroxamate, hydroxamic acid, heteroaryloxy, aminoalkyl, aminoaryl, aminoheteroaryl, thioalkyl, thioaryl or thioheteroaryl, which may optionally be further substituted,
with the proviso that XR₂, or XR₃ or XR₄ or XR₅ is not NH₂,
with the further proviso that not more than one of R₂ to R₅ is hydrogen,
where the group X is NR^{A} and R^{A} is not hydrogen, the groups R^{A} and the corresponding group R₂ to R₅ may combine to form a cycle;
or a pharmaceutically acceptable salt thereof, to a subject in need.

9. The method of claim 8 in which the compound is selected from the group defined by formula II: 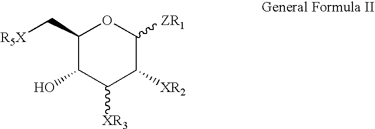
or a pharmaceutically acceptable salt thereof;
wherein R₁, R₂, R₃, R₅, Z and X are defined as in General Formula I.

10. The method of claim 8 in which the compound is selected from the group defined by formula III: 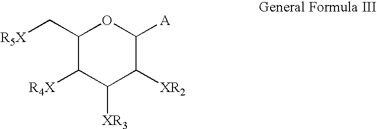
or a pharmaceutically acceptable salt thereof;
wherein A is defined as hydrogen, SR₁, or OR₁, where R₁ is defined as in General Formula I, and X and R₂ to R₅ are defined as in General Formula I.

11. The method of claim 8 in which the compound is selected from the group defined by formula IV: 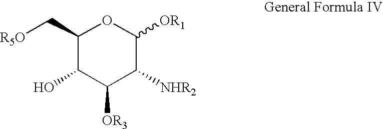
or a pharmaceutically acceptable salt thereof;
wherein R₁-R₃ and R₅ are defined as in General Formula I.

12. The method of claim 8 in which the compound is selected from the group defined by formula V: 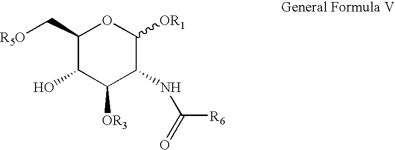
or a pharmaceutically acceptable salt thereof;
wherein R¹, R₃, R₅ and R₆ are independently selected from the group comprising an alkyl, alkenyl, alkynyl, heteroalkyl, aryl, heteroaryl, arylalkyl or heteroarylalkyl substituent of 1 to 20 atoms, which is optionally substituted, and can be branched or linear wherein substituents include but are not limited to OH, NO, NO₂, NH₂, N₃, halogen, CF₃, CHF₂, CH₂F, nitrile, alkoxy, aryloxy, amidine, guanidiniums, carboxylic acid, carboxylic acid ester, carboxylic acid amide, aryl, cycloalkyl, heteroalkyl, heteroaryl, aminoalkyl, aminodialkyl, aminotrialkyl, aminoacyl, carbonyl, substituted or unsubstituted imine, sulfate, sulfonate, sulfonamide, phosphate, phosphoramide, hydrazide, hydroxamate, hydroxamic acid, heteroaryloxy, aminoalkyl, aminoaryl, aminoheteroaryl, thioalkyl, thioaryl or thioheteroaryl, which may optionally be further substituted, with the proviso that one of the groups R₁, R₃, R₅, or R₆ contains an acidic substituent including but not limited to. a carboxylate, a sulfonate, a phosphate, a hydroxamate, a phenol; or an adic mimetic substituent including but not limited to: a tetrazole, an amide, an ester, a sulfonamide, a phosphoramide; and any of the remaining groups R₁, R₃, R₅, or R₆ contains a basic substituent including but not limited to: a primary amine, a secondary amine, a tertiary amine, a quaternary amine, an amidine, a guanidinium group, an imidazole group, a triazole group.

13. The method according to claim 8, wherein the disease or condition is selected from the group consisting of diabetes, diabetic retinopathy, aged related macular degeneration, multiple sclerosis, asthma, arthritis, Crohn's disease and colitis, cancer, tumour metastasis, tumour growth, angiogenesis, neovascularisation, cardiovascular disorder, wound healing, thrombosis and osteoporosis, and related diseases or conditions.

14. A compound of formula VI: 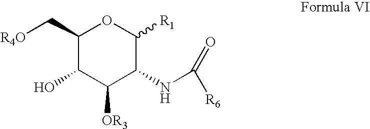
or a pharmaceutically acceptable salt thereof;
wherein R₁ is selected from the group consisting of alkyl, hydroxy, alkoxy, aryloxy, arylalkyloxy, heteroaryloxy or benzyloxy; R₆ is alkyl, aryl, heteroaryl; R₃ is alkyl, aryl or arylalkyl; R₄ is aryl, arylalkyl; and wherein each of R₁, R₃, R₄ and R₆ may be further optionally substituted; and wherein the compound is effective to inhibit or effect the activity of an integrin receptor.

15. The compound according to claim 14 wherein R₁ is selected from the group consisting of methoxy, ethoxy, hydroxyl, benzyloxy and phenoxy.

16. The compound of claim 14 in which one of the groups R₁, R₃, R₄ or R₆ is substituted with a carboxylic acid or a carboxylic acid ester or a carboxylate anion or a carboxylate salt.

17. The compound of claim 14 in which one of the groups R₃ or R₄ or R₆ is selected from the group consisting of hydroxy, methyl, ethyl, phenyl, benzyl, piperidine, triazole, tetrazole, imidazole, 4-aminomethylcyclohexane, carboxyphenyl, carboxybenzyl, chlorophenyl, bromobenzyl, amino phenyl, carboxymethylene, carboxyethylene, ethylguinidine, 4-guanidomethylphenyl, 3,5-diaminophenyl and (3,5-diaminophenyl)bis-formamide.

18. A compound according to claim 14 wherein the compound is selected from the following compounds:

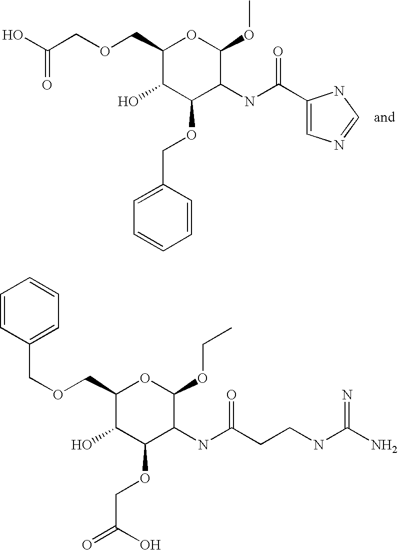

19-20. (canceled)

21. A method of inhibiting or effecting the activity of an integrin receptor which comprises contacting an integrin with a compound of the formula: 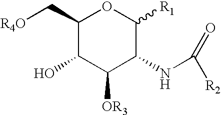
wherein the compounds are selected from the compounds defined in the following Table:

| R1 | R2a | R3 | R4 |
|---|---|---|---|
| OH | -(3-aminophenyl) | -(4-bromobenzyl) | —CH2-CO2H |
| OH | -(3-aminophenyl) | -(4-bromobenzyl) | —CH2-CO2H |
| OMe | -(4-carboxyphenyl) | -(4-bromobenzyl) | -(3-aminobenzyl) |
| OMe | -(4-carboxyphenyl) | -(4-bromobenzyl) | -(3-aminobenzyl) |
| OMe | -(3-aminophenyl) | —CH2-CO2H | -Bn |
| OMe | -(3-aminophenyl) | —CH2-CO2H | -Bn |
| OMe | -(3-aminophenyl) | —CH2-CO2H | -(4-bromobenzyl) |
| OMe | -(3-aminophenyl) | —CH2-CO2H | -(4-bromobenzyl) |
| OMe | -(4-chlorophenyl) | —CH2-CO2H | -(3-aminobenzyl) |
| OMe | -(4-chlorophenyl) | —CH2-CO2H | -(3-aminobenzyl) |
| OMe | —CH2-CH2-NH—C—(═NH)—NH2 | -(4-carboxybenzyl) | -Bn |
| OMe | —CH2-CH2-NH—C—(═NH)—NH2 | -(4-carboxybenzyl) | -Bn |
| OMe | —CH2-CH2-NH—C—(═NH)—NH2 | -(4-carboxybenzyl) | -Bn |
| | | methyl ester | |
| OMe | —CH2-CH2-NH—C—(═NH)—NH2 | -(4-carboxybenzyl) | -Bn |
| | | methyl ester | |
| OMe | —CH2-CH2-NH—C—(═NH)—NH2 | -(4-carboxybenzyl) | -(4-bromobenzyl) |
| OMe | —CH2-CH2-NH—C—(═NH)—NH2 | -(4-carboxybenzyl) | -(4-bromobenzyl) |
| OMe | —CH2-CH2-NH—C—(═NH)—NH2 | -(4-carboxybenzyl) | -(4-bromobenzyl) |
| | | methyl ester | |
| OMe | -phenyl | -(4-carboxybenzyl) | -(3-aminobenzyl) |
| OMe | -phenyl | -(4-carboxybenzyl) | -(3-aminobenzyl) |
| OMe | —CH2-CH2-CO2H | -(3-aminobenzyl) | -Bn |
| OMe | —CH2-CH2-CO2H | -(3-aminobenzyl) | -Bn |
| OH | —CH2-CH2-CO2H | -(3-aminobenzyl) | -Bn |
| OH | —CH2-CH2-CO2H | -(3-aminobenzyl) | -Bn |
| OMe | —CH2-CH2-CO2H | -(3-aminobenzyl) | -(4-bromobenzyl) |
| OMe | —CH2-CH2-CO2H | -(3-aminobenzyl) | -(4-bromobenzyl) |
| OH | —CH2-CH2-CO2H | -(3-aminobenzyl) | -(4-bromobenzyl) |
| OMe | -phenyl | -(3-aminobenzyl) | —CH2-CO2H |
| OMe | -phenyl | -(3-aminobenzyl) | —CH2-CO2H |
| OH | -phenyl | -(3-aminobenzyl) | —CH2-CO2H |
| OMe | -(4-chlorophenyl) | -(3-aminobenzyl) | -(4-carboxybenzyl) |
| OMe | -(4-chlorophenyl) | -(3-aminobenzyl) | -(4-carboxybenzyl) |
| OEt | -(3-aminophenyl) | -Bn | —CH2-CO2H |
| OH | -(3-aminophenyl) | -Bn | —CH2-CO2H |
| OH | -(3-aminophenyl) | -Bn | —CH2-CO2H |
| OEt | -(3-aminophenyl) | -Bn | -(4-carboxybenzyl) |
| OEt | -(4-carboxyphenyl) | -Bn | -(3-aminobenzyl) |
| OEt | -(4-carboxyphenyl) | -Bn | -(3-aminobenzyl) |
| OEt | -(3-aminophenyl) | -(4-bromobenzyl) | —CH2-CO2H |
| OEt | -(3-aminophenyl) | -(4-bromobenzyl) | —CH2-CO2H |
| OEt | -(3-aminophenyl) | -(4-bromobenzyl) | -(4-carboxybenzyl) |
| OEt | -(3-aminophenyl) | -(4-bromobenzyl) | -(4-carboxybenzyl) |
| OH | -(3-aminophenyl) | -(4-bromobenzyl) | -(4-carboxybenzyl) |
| OH | -(3-aminophenyl) | -(4-bromobenzyl) | -(4-carboxybenzyl) |
| OEt | —CH2-CH2-CO2H | -(4-bromobenzyl) | -(3-aminobenzyl) |
| OEt | —CH2-CH2-CO2H | -(4-bromobenzyl) | -(3-aminobenzyl) |
| OH | —CH2-CH2-CO2H | -(4-bromobenzyl) | -(3-aminobenzyl) |
| OH | —CH2-CH2-CO2H | -(4-bromobenzyl) | -(3-aminobenzyl) |
| OEt | —CH2-CH2-NH—C—(═NH)—NH2 | —CH2-CO2H | -Bn |
| OEt | —CH2-CH2-NH—C—(═NH)—NH2 | —CH2-CO2H | -Bn |
| OEt | —CH2-CH2-NH—C—(═NH)—NH2 | —CH2-CO2H | -(4-bromobenzyl) |
| OEt | —CH2-CH2-NH—C—(═NH)—NH2 | —CH2-CO2H | -(4-bromobenzyl) |
| OEt | —CH2-CH2-NH—C—(═NH)—NH2 | —CH2-CO2H | -(4-bromobenzyl) |
| OEt | —CH2-CH2-NH—C—(═NH)—NH2 | —CH2-CO2H | -(4-bromobenzyl) |
| OEt | -phenyl | —CH2-CO2H | -(3-aminobenzyl) |
| OEt | -phenyl | —CH2-CO2H | -(3-aminobenzyl) |
| H | -phenyl | —CH2-CO2H | -(3-aminobenzyl) |
| OEt | —CH2-CH2-NH—C—(═NH)—NH2 | -(4-carboxybenzyl) | -Bn |
| OEt | —CH2-CH2-NH—C—(═NH)—NH2 | -(4-carboxybenzyl) | -Bn |
| OH | —CH2-CH2-NH—C—(═NH)—NH2 | -(4-carboxybenzyl) | -Bn |
| OEt | —CH2-CH2-NH—C—(═NH)—NH2 | -(4-carboxybenzyl) | -(4-bromobenzyl) |
| OEt | —CH2-CH2-NH—C—(═NH)—NH2 | -(4-carboxybenzyl) | -(4-bromobenzyl) |
| OEt | —CH2-CH2-NH—C—(═NH)—NH2 | -(4-carboxybenzyl) | -(4-bromobenzyl) |
| OH | —CH2-CH2-NH—C—(═NH)—NH2 | -(4-carboxybenzyl) | -(4-bromobenzyl) |
| OEt | -(4-chlorophenyl) | -(4-carboxybenzyl) | -(3-aminobenzyl) |
| OEt | -(4-chlorophenyl) | -(4-carboxybenzyl) | -(3-aminobenzyl) |
| OH | -(4-chlorophenyl) | -(4-carboxybenzyl) | -(3-aminobenzyl) |
| OH | -(4-chlorophenyl) | -(4-carboxybenzyl) | -(3-aminobenzyl) |
| OEt | -(4-carboxyphenyl) | -(3-aminobenzyl) | -Bn |
| OEt | -(4-carboxyphenyl) | -(3-aminobenzyl) | -Bn |
| OH | -(4-carboxyphenyl) | -(3-aminobenzyl) | -Bn |
| OH | -(4-carboxyphenyl) | -(3-aminobenzyl) | -Bn |
| OEt | -(4-carboxyphenyl) | -(3-aminobenzyl) | -(4-bromobenzyl) |
| OEt | -(4-carboxyphenyl) | -(3-aminobenzyl) | -(4-bromobenzyl) |
| OH | -(4-carboxyphenyl) | -(3-aminobenzyl) | -(4-bromobenzyl) |
| OH | -(4-carboxyphenyl) | -(3-aminobenzyl) | -(4-bromobenzyl) |
| OEt | -(4-chlorophenyl) | -(3-aminobenzyl) | —CH2-CO2H |
| OEt | -(4-chlorophenyl) | -(3-aminobenzyl) | —CH2-CO2H |
| OEt | -(4-chlorophenyl) | -(3-aminobenzyl) | -(4-carboxybenzyl) |
| OEt | -(4-chlorophenyl) | -(3-aminobenzyl) | -(4-carboxybenzyl) |
| H | -(4-chlorophenyl) | -(3-aminobenzyl) | —CH2-CO2H |
| OH | -(4-chlorophenyl) | -(3-aminobenzyl) | -(4-carboxybenzyl) |
| OH | -(4-chlorophenyl) | -(3-aminobenzyl) | -(4-carboxybenzyl) |
| OH | -(4-chlorophenyl) | -(3-aminobenzyl) | -(4-carboxybenzyl) |
| OBn | -(3-aminophenyl) | -Et | —CH2-CO2H |
| OBn | -(3-aminophenyl) | -Et | —CH2-CO2H |
| OBn | —CH2-CH2-NH—C—(═NH)—NH2 | -Et | -(4-carboxybenzyl) |
| OBn | —CH2-CH2-NH—C—(═NH)—NH2 | -Et | -(4-carboxybenzyl) |
| OBn | —CH2-CH2-CO2H | -Et | -(3-aminobenzyl) |
| OBn | —CH2-CH2-CO2H | -Et | -(3-aminobenzyl) |
| OBn | —CH2-CH2-NH—C—(═NH)—NH2 | —CH2-CO2H | -Me |
| OBn | —CH2-CH2-NH—C—(═NH)—NH2 | —CH2-CO2H | -Me |
| OBn | —CH2-CH2-NH—C—(═NH)—NH2 | —CH2-CO2H | -Et |
| OBn | —CH2-CH2-NH—C—(═NH)—NH2 | —CH2-CO2H | -Et |
| OBn | —CH2-CH2-NH—C—(═NH)—NH2 | —CH2-CO2H | H |
| OBn | -Me | —CH2-CO2H | -(3-aminobenzyl) |
| OBn | -Me | —CH2-CO2H | -(3-aminobenzyl) |
| OBn | —CH2-CH2-NH—C—(═NH)—NH2 | -(4-carboxybenzyl) | -Me |
| OBn | —CH2-CH2-NH—C—(═NH)—NH2 | -(4-carboxybenzyl) | -Me |
| OBn | -(3-aminophenyl) | -(4-carboxybenzyl) | -Et |
| OBn | -(3-aminophenyl) | -(4-carboxybenzyl) | -Et |
| OBn | -(3-aminophenyl) | -(4-carboxybenzyl) | H |
| OBn | -(3-aminophenyl) | -(4-carboxybenzyl) | H |
| OBn | -Et | -(4-carboxybenzyl) | -(3-aminobenzyl) |
| OBn | -Et | -(4-carboxybenzyl) | -(3-aminobenzyl) |
| OBn | -(4-carboxyphenyl) | -(3-aminobenzyl) | -Me |
| OBn | -(4-carboxyphenyl) | -(3-aminobenzyl) | -Me |
| OBn | -(4-carboxyphenyl) | -(3-aminobenzyl) | -Et |
| OBn | -(4-carboxyphenyl) | -(3-aminobenzyl) | -Et |
| OBn | -(4-carboxyphenyl) | -(3-aminobenzyl) | H |
| OBn | -Et | -(3-aminobenzyl) | —CH2-CO2H |
| OBn | -Et | -(3-aminobenzyl) | -(4-carboxybenzyl) |
| OBn | -Et | -(3-aminobenzyl) | -(4-carboxybenzyl) |
| O—CH2-CH2-NH—C(═NH)—NH2 | -Me | -Bn | —CH2-CO2H |
| O—CH2-CH2-NH—C(═NH)—NH2 | -phenyl | -(4-carboxybenzyl) | -Me |
| O—CH2-CH2-NH—C(═NH)—NH2 | -phenyl | -(4-carboxybenzyl) | -Me |
| OEt | —CH2-NH2 | -Bn | —CH2-CO2H |
| OEt | —CH2-NH2 | -Bn | —CH2-CO2H |
| OEt | —CH2-NH—C—(═NH)—NH2 | -Bn | —CH2-CO2H |
| OEt | —CH2-NH—C—(═NH)—NH2 | -Bn | —CH2-CO2H |
| OEt | —CH2-CH2-NH2 | -Bn | —CH2-CO2H |
| OEt | —CH2-CH2-NH2 | -Bn | —CH2-CO2H |
| OEt | —CH2-CH2-NH—C—(═NH)—NH2 | -Bn | —CH2-CO2H |
| OMe | —CH2-CH2-CH2-NH2 | -Bn | —CH2-CO2H |
| OMe | —CH2-CH2-CH2-NH2 | -Bn | —CH2-CO2H |
| OMe | —CH2-CH2-CH2-NH—C—(═NH)—NH2 | -Bn | —CH2-CO2H |
| OMe | —CH2-CH2-CH2-NH—C—(═NH)—NH2 | -Bn | —CH2-CO2-CH3 |
| OMe | -(4-aminomethyl)phenyl | -Bn | —CH2-CO2H |
| OMe | -(4-guanadinomethyl)phenyl | -Bn | —CH2-CO2H |
| OMe | -(4-guanadinomethyl)phenyl | -Bn | —CH2-CO2H |
| OMe | -(3,5-diaminophenyl) | -Bn | —CH2-CO2H |
| OMe | -(3,5-diaminophenyl) | -Bn | —CH2-CO2H |
| OMe | -3′-imidazole | -Bn | —CH2-CO2H |
| OMe | -3′-imidazole | -Bn | —CH2-CO2H |
| OMe | -4′-piperidine | -Bn | —CH2-CO2H |
| OMe | -4′-piperidine | -Bn | —CH2-CO2H |
| OMe | -4′-pipeiridine | -Bn | —CH2-CO2H |
| OMe | -4-aminomethylcyclohexane | -Bn | —CH2-CO2H |
| OMe | -4-aminomethylcyclohexane | -Bn | —CH2-CO2H |
| H | -(4-carboxyphenyl) | -(4-bromobenzyl) | —CH2-CO2H |
| H | -(4-carboxyphenyl) | -(3-aminobenzyl) | -Bn |
| H | -(4-chlorophenyl) | -(3-aminobenzyl) | -(4-carboxybenzyl) |
| H | -(3-aminophenyl) | -Et | —CH2-CO2H |
| H | —CH2-CH2-NH—C—(═NH)—NH2 | -Et | -(4-carboxybenzyl) |
| H | -(4-guanadinomethyl)phenyl | -Bn | —CH2-CO2H |
| H | -(3,5-diaminophenyl) bis formamide | -Bn | —CH2-CO2H |

or a pharmaceutically acceptable salt thereof.

22. A composition comprising a compound of claim 14 or a pharmaceutically acceptable salt thereof.
